(19) United States
(12) Patent Application Publication
(10) Pub. No.: US 2005/0281898 A1
(43) Pub. Date: Dec. 22, 2005

(54) Anti-tumor agent, beverages and foods using the same, and a process for manufacturing the anti-tumor agent

(75) Inventors: Tsuyoshi Sugiyama, Odawara-City (JP); Tatsuhiko Matsuo, Nagoya (JP); Kazuyuki Futakuchi, Suita-City (JP)

Correspondence Address: SENNIGER POWERS, ONE METROPOLITAN SQUARE, 16TH FLOOR, ST LOUIS, MO, 63102, US

(73) Assignees: Oto Corporation, Odawara-City (JP); Validux Co., Ltd., Nagoya (JP)

(21) Appl. No.: 11/026,088

(22) Filed: Dec. 30, 2004

(30) Foreign Application Priority Data
Jun. 18, 2004 (JP) 2004-181879

(51) Int. Cl. A61K 35/78

(52) U.S. Cl. 424/750; 424/757

(57) ABSTRACT

Disclosed are an anti-tumor agent, beverages and foods using the same and a process for manufacturing the anti-tumor agent. The anti-tumor agent according to the present invention exhibits specific effects on human tumor cells A4573 and mouse melanoma B16 cells in vitro. The anti-tumor agent exhibits various immune activities in addition to immune activity against tumors. Beverages and foods containing said anti-tumor agent can be manufactured as health food or the like.

### REFERENCE TO RELATED APPLICATIONS

This application claims priority from Japanese Patent Application No. 2004-181879, filed Jun. 18, 2004, which is hereby incorporated by reference in its entirety.

### FIELD OF THE INVENTION

Generally, the present invention relates to an anti-tumor agent and a process of manufacturing the same, in which such agent may be utilized in beverages and foods as an ingredient. More specifically, the present invention relates to (1) an anti-tumor agent obtained from fermented materials such as rice germ, wheat germ and soybean, (2) beverages and foods containing such anti-tumor agent, and (3) a process of manufacturing said anti-tumor agent. The present invention is widely applied in health foods, treatments of diseases, and the like.

### BACKGROUND OF THE INVENTION

Conventionally, various immune adjuvants from synthetic compounds have been suggested. However, those from natural origins are desirable since they show no adverse effects even in long-term use. It has been traditionally known that immune adjuvants and anti-tumor agents originated from nature (see Japanese Patent Laid-Open (Hei)6-9421, Japanese Patent Laid-Open 2000-224970, and Japanese Patent Laid-Open 2003-335695). In addition, the present inventors have already suggested an enzyme food that purifies turbid blood (see Japanese Patent Laid-Open 2001-120203).

However, an anti-tumor agent from natural origin, which (1) makes erythrocyte aggregation causing cerebral thrombosis, brain infarct and pulmonary thromboembolism become individually separated and substantially circular shaped and (2) repairs erythrocytes of abnormal shapes into normal erythrocytes in a short amount of time to provide the effect of treating diseases resulting from such abnormal shapes of erythrocytes, is not known in the prior art. The present inventors have performed intensive studies and found from observation by microscope according to Live Blood Analysis that erythrocyte aggregations have become individually separated and substantially circular in shaped by means of a certain fermented material, while the leukocytes became enlarged and the motion activated. As a result, they discovered that the fermented material can prevent cerebral thrombosis, brain infarct and pulmonary thromboembolism, as well as increase immunity. The present inventors completed the anti-tumor agent of the present invention on the basis of such discovery. Further, the anti-tumor agent of the present invention repairs erythrocytes of abnormal shapes into those of normal shapes in a short amount of time so as to provide the effect of treating various diseases.

In addition, the present inventors found anti-tumor effect of the present anti-tumor agent using in vitro testing of rats. More specifically, it was discovered by means of Live Blood Analysis that the anti-tumor agent of the present invention exhibits extensive effects of increasing immunity, as well as immunity against tumors, in order to provide effective results on various types of diseases.

### SUMMARY OF THE INVENTION

The present invention provides an anti-tumor agent obtained from fermented materials such as rice germ, wheat germ and soybean. The present invention further provides beverages and foods that contain such anti-tumor agent as an ingredient, as well as a process for manufacturing said anti-tumor agent. The anti-tumor agent according to the present invention (1) increases immunity against tumors, (2) increases activity of leukocytes to augment various immunities against those other than tumors, (3) dissolves uric acid crystals or the like in blood, and (4) repairs abnormal erythrocytes into those of normal shape to give the effect of treating various diseases owing to the erythrocytes of abnormal shape.

The present invention provides:
1. An anti-tumor agent obtained by fermenting raw material containing one or more substance(s) selected from the group consisting of germs of wheat, germs of soybean and germs of rice by using one or more microbe(s) selected from the group consisting of leavens, yeasts and lactobacilli.
2. An anti-tumor agent according to clause 1, in which such agent makes one or more types of erythrocytes, having existed before internal use, selected from the group consisting of erythrocyte aggregation, acanthocytes, target-cell type erythrocytes, erythrocytes connections, echinocytes, microcytes, macrocytes, hemolytic erythrocytes (hemolysis), erythrocytes arranged in chain (rouleau), ovalocytes, and free radical damaged erythrocytes (poikilocytosis) into, after internal use of the agent, erythrocytes of substantially circular shape with individual erythrocytes separated.
3. An anti-tumor agent according to clause 1 or 2, in which such agent makes the diameter of leukocytes in blood, after internal use, as large as 2.2 times or more of the diameter of normal erythrocytes.
4. An anti-tumor agent according to any one of clauses 1 to 3, which removes uric acid crystals in blood.
5. An anti-tumor agent according to any one of clauses 1 to 4, which exhibits one or more additional effect(s) selected from the group consisting of alleviating arthralgia, improving liver function, reducing uterine myoma, reducing cancer cells, decreasing cholesterol, decreasing triglyceride and treating atopic dermatitis.
6. Beverages and foods containing and using the anti-tumor agent according to any one of clauses 1 to 5.
7. A process for manufacturing the anti-tumor agent described in any one of clauses 1 to 5, which comprises steps of (1) steaming one or more substance(s) selected from the group consisting of germs of wheat, germs of soybean and germs of rice at 50° C. to 150° C., or steaming the same after roasting the substance(s) at 50° C. to 150° C.; and (2) fermenting the resulting steamed product by using one or more microbes selected from the group consisting of leavens, yeasts and lactobacilli.
8. A process according to clause 7, which comprises an additional step of treating the resulting fermented material with hot water at 60° C. to 100° C.

### BRIEF DESCRIPTION OF DRAWINGS

FIG. 1 shows anti-tumor effects associated with the anti-tumor agent of the present invention against human A4573 Ewing's sarcoma.

FIG. 2 shows anti-tumor effects associated with the anti-tumor agent of the present invention against B16 mouse melanoma cells.

FIG. 3 shows change of body weight in mice inoculated with the anti-tumor agent of the present invention.

FIG. 4 shows proliferation of B16 cells subcutaneously inoculated in mice that had ingested the anti-tumor agent of the present invention.

FIG. 5 shows days of survival of mice that ingested the anti-tumor agent of the present invention after subcutaneous inoculation of B16 cells.

FIG. 6 shows change of body weight in mice that ingested the anti-tumor agent of the present invention after inoculation of B16 cells in caudal vein.

FIG. 7 shows the number of cells of pulmonary metastasis in mice inoculated with B16 cells in the caudal vein after 19 days from the inoculation.

FIG. 8 shows the number of cells of pulmonary metastasis in mice inoculated with B16 cells in the caudal vein after 22 days from the inoculation.

FIG. 9 is a duplicate copy of micrograph showing the erythrocyte aggregation with existence of acanthocytes.

FIG. 10 is a duplicate copy of micrograph showing individual hemocytes separated as substantially circular shapes with no existence of acanthocytes.

FIG. 11 is a duplicate copy of micrograph wherein the erythrocytes in blood are acanthocytes.

FIG. 12 is a duplicate copy of micrograph wherein individual erythrocytes are separated as substantially circular shapes in blood with no existence of acanthocytes so as to enlarge the leukocytes three times or more the size of erythrocytes.

FIG. 13 is a duplicate copy of micrograph showing the erythrocytes in blood appearing as target cells and ovalocytes.

FIG. 14 is a duplicate copy of micrograph showing the target cell type erythrocytes and ovalocytes in blood that became normal erythrocytes.

FIG. 15 is a duplicate copy of micrograph showing the erythrocytes being aggregated with existence of uric acid crystals and fungal forms.

FIG. 16 is a duplicate copy of micrograph wherein individual erythrocytes in blood are separated as substantially circular shapes, and wherein uric acid crystals are dissolved therein with no existence of fungal forms while the leukocytes are enlarged.

FIG. 17 is a duplicate copy of micrograph wherein erythrocyte aggregation is prominent in blood, and wherein the leukocytes are so small that they are surrounded by the erythrocytes.

FIG. 18 is a duplicate copy of micrograph wherein erythrocyte aggregation in blood disappeared, in which individual erythrocytes were separated as substantially circular shapes, to portray a normal state.

FIG. 19 is a duplicate copy of micrograph showing the state wherein erythrocytes in blood are aggregated and three leukocytes (granulocytes) are present in one image.

FIG. 20 is a duplicate copy of micrograph wherein individual erythrocytes in blood are separated as substantially circular shapes and B cells of lymphocytes are enlarged to show activated state.

### DETAILED DESCRIPTION OF THE PREFERRED EMBODIMENTS

The present invention is described below in more detail.

Anti-Tumor Agent (Fermented Material)

The types of germs of wheat, germs of soybean and germs of rice used as raw materials for the anti-tumor agent of the present invention are not specifically restricted. For example, all types of germs such as those from barley, wheat, adlay, rye and oat may be used as the germs of wheat. A combination of two or more types of such germs of wheat may also be used. At present, about 200 kinds of soybeans are known, and any germs from these kinds of soybeans may be used. Germs from any kind of bean, such as black bean, red-wave black bean and green bean may be used. A combination of two or more kinds of these beans may be used. For the germs of rice, germs of any kind of rice may be used. Germs may be from various kinds of rice such as long-grain type, medium-grain type or short-grain type. A combination of two or more kinds of these may be also used. Germs of wheat, germs of soybean and germs of rice may be used individually or in combination. The ratio of those raw materials can be appropriately selected. For example, the ratio of germs between wheat germs, soybean germs and rice germs may be 1:1:1. In addition, two raw materials may be used, or one raw material may be used alone. Among the ratios of those raw materials, the ratio of 5:5:4 is preferably used between wheat germs, soybean germs and rice germs.

To the raw materials mentioned above, one or more substance(s) selected from the group consisting of adlay, soybean and whole-rice may be additionally incorporated. If the substance(s) are incorporated, the fermenting efficiency of the raw material increases, and lowering of the molecular weight is facilitated so that the anti-tumor agent can be easily absorbed in vivo. The substance(s) may be added as is, or after being formed in a crushed or pulverized state. The proportion of the substance(s) is preferably from 5% to 60% by weight, more preferably from 5% to 50% by weight, and most preferably from 10% to 40% by weight based on 100% by weight of total raw material after the addition of adlay or the like to the germs. In particular, the fermenting efficiency becomes higher when the proportion is within the range described above.

The raw material is fermented by the use of one or more microbe(s) selected from leavens, yeasts and lactobacilli. The type of leavens is not particularly restricted as long as it can ferment the raw material. Leavens such as Aspergillus oryzae and Aspergillus sojae may be used. Only one kind of leaven can be used, or a combination of two or more kinds of leavens can be used. Among those leavens, Aspergillus oryzae is preferably used.

The type of yeast is not particularly restricted as long as it can ferment the raw material. Saccharomyces cerevisiae, Saccharomyces rouxii, or the like may be used. Only one kind of yeast can be used, or a combination of two or more kinds of yeasts can be used. Among those yeasts, Saccharomyces cerevisiae is preferably used.

The type of lactobacillus is not particularly restricted as long as it can ferment the raw material. Lactobacilli such as Lactobacillus plantarum, Lactobacillus salivarius, Lactobacillus brevis, Lactobacillus casei, Lactobacillus acidophilus, Lactobacillus helveticus, Streptococcus lactis, Streptococcus thermophilus and Enterococcus faecalis may be used. Only one kind of lactobacillus can be used, or a combination of two or more kinds of lactobacilli can be used. Among those lactobacilli, Lactobacillus plantarum, Lactobacillus salivarius, Lactobacillus brevis, Lactobacillus casei and Lactobacillus acidophilus are preferably used.

Two or more of the leavens, yeasts and lactobacilli may be cultured or fermented together.

The raw material may be steamed at 50° C. to 150° C. Preferably, it is steamed at 60° C. to 140° C., more preferably at 70° C. to 130° C., and most preferably at 80° C. to 120° C. At a temperature below 50° C., starch or protein is not sufficiently denatured so as to be fermented appropriately. At a temperature above 150° C., the active component, which subsequently becomes an anti-tumor agent in the germs, is apt to be disintegrated.

The method of steaming is not particularly restricted. Though various conventional means may be employed, an autoclave is typically used. The duration of steaming is not particularly restricted, but preferably is from 30 to 500 minutes, more preferably from 50 to 480 minutes, and most preferably from 70 to 460 minutes. If the duration is less than 30 minutes, steaming is apt to be insufficient. If the duration is more than 500 minutes, essential components may be disintegrated.

Before the steaming process, the raw material may be roasted. The method of roasting is not particularly restricted, but a known process may be used. Among the known processes, roasting by the use of far infrared is preferably used. The duration of roasting is preferably from 30 to 500 minutes, more preferably from 50 to 480 minutes, and most preferably from 70 to 460 minutes. If the duration is less than 30 minutes, insufficient roasting can lower the efficiency of fermentation. If the duration is more than 500 minutes, essential components may be disintegrated.

The raw material steamed, or steamed after roasting, is usually cooled to a temperature preferably from 20° C. to 50° C., more preferably from 23° C. to 45° C., and most preferably from 26° C. to 40° C. After mixing with leaven or the like as described above, the cooled raw material is fermented in a fermentation chamber preferably at 24° C. to 42° C., more preferably at 25° C. to 41° C., and most preferably at 26° C. to 40° C. It is fermented preferably for 24 to 72 hours, more preferably for 28 to 68 hours, and most preferably for 32 to 64 hours. The combination of temperature and duration of fermentation is preferably at 24° C. to 42° C. for 24 to 72 hours, more preferably at 25° C. to 41° C. for 28 to 68 hours, and most preferably at 26° C. to 40° C. for 32 to 64 hours.

Thereafter, the substance is removed from the fermentation chamber and dried to obtain the fermented material (anti-tumor agent). For drying, known methods including hot air drying, drying under reduced pressure and lyophilzation may be employed. Among them, hot air drying is preferably used, and drying is performed at 35° C. to 45° C. for 10 to 20 hours. Then, if necessary, sterilization may be performed. The anti-tumor agent may be made as powder, granules, tablets or capsules depending upon the intended purpose.

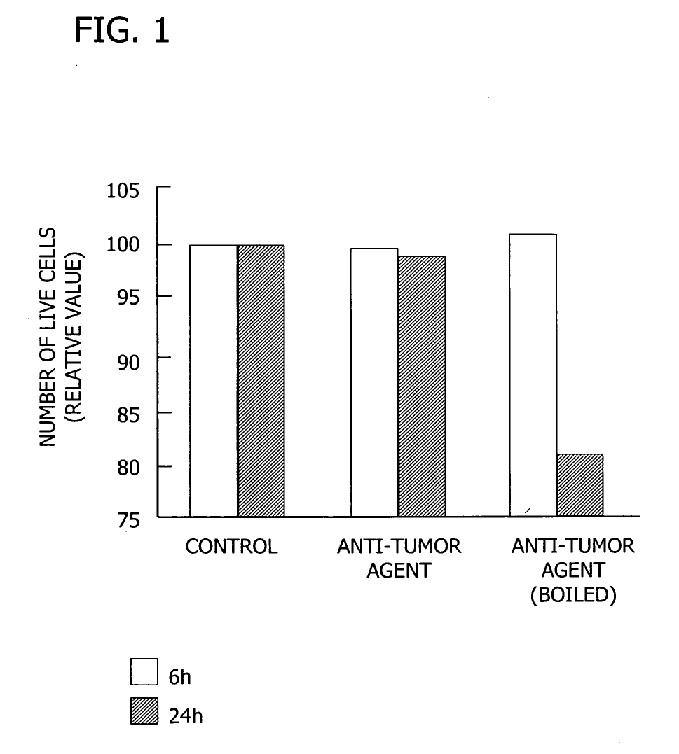

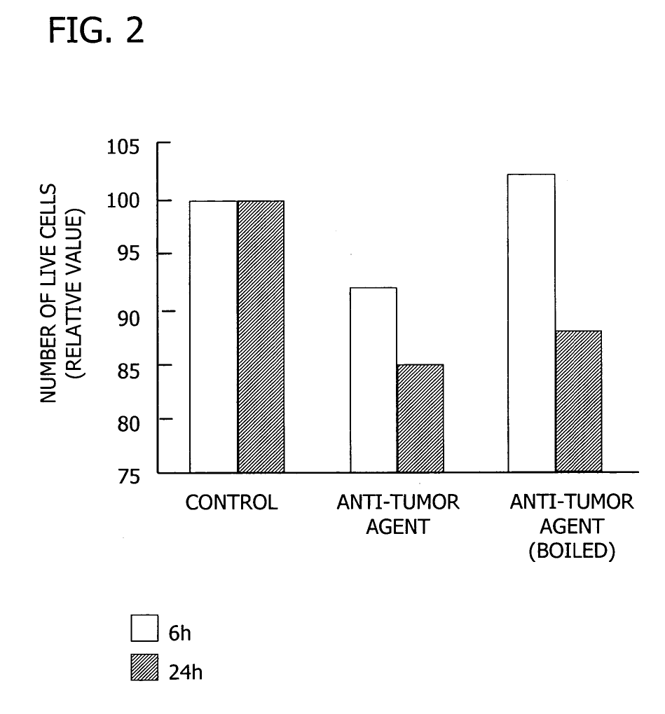

Further, the fermented material can be treated with hot water. This treatment is performed preferably at 60 to 100° C., more preferably at 65 to 98° C., and most preferably at 70 to 96° C. Such treatment is preferably for 5 to 35 minutes, more preferably for 10 to 30 minutes, and most preferably for 15 to 25 minutes. Occasionally, the anti-tumor agent, when treated with hot water, exhibits superior effects in comparison to the untreated agent (see FIGS. 1 and 2).

To the culture solution of human A4573 Ewing's sarcoma cultivated in a 96-well plate, the anti-tumor agent of the present invention having a concentration of 1 mg/ml is added. After 24 hours, the reagent for measuring the live cells (tetrazolium salt: manufactured by Nakarai Tesk) is added and the number of live cells is measured by examining the color reaction after one hour by using microplate reader (450 nm). As a result, the number of live cells when treated by the anti-tumor agent according to the present invention which was hot-water treated can be lowered to preferably 90% or less, more preferably 88% or less, and most preferably 85% or less of that of live cells without the addition of the anti-tumor agent according to the present invention (hereinafter, referred to as “the control group”).

Similarly, to the culture solution of mouse melanoma B16 cultivated in a 96-well plate, the anti-tumor agent of the present invention having a concentration of 1 mg/ml is added. After 24 hours, the reagent for measuring the live cells (tetrazolium salt: manufactured by Nakarai Tesk) is added and the number of live cells is measured by examining the color reaction after one hour by using microplate reader (450 nm). As a result, the number of live cells when treated by the anti-tumor agent according to the present invention which was not treated with hot water can be lowered to preferably 90% or less, more preferably 88% or less, and most preferably 85% or less of that of live cells of the control group. In case of using the anti-tumor agent which was hot-water treated, the number of live cells can be preferably 90% or less, more preferably 89% or less, and most preferably 88% or less of that of the control group.

When 5-week old C57BL6 mice are fed with solid feed prepared as containing 0.4% of the anti-tumor agent, and B16 cells (5×105 cells/mouse) is inoculated in the caudal vein after two weeks from the feeding, the volume percentage of the tumor in the group inoculated with the anti-tumor agent after 22 days from inoculation can be lowered to preferably 65% by volume or less, more preferably 60% by volume or less, and most preferably 55% by volume of the tumor in the control group.

When their body weights were measured after 22 days from the inoculation, the ratio of average body weight of the anti-tumor agent administered group to that of the control group can be preferably at least 104%, more preferably at least 105%, and most preferably at least 106%.

In addition, the number of cells of pulmonary metastasis after 22 days from inoculation can be lowered to preferably 45% or less, more preferably 40% or less, and most preferably 35% or less as compared to that of the control group.

When observed by microscopy according to Live Blood Analysis after administration of the anti-tumor agent (fermented material obtained from the process described above), each erythrocyte in the blood preferably becomes substantially circular shape, with individual erythrocytes being separated. The Live Blood Analysis is LBA (Live Blood Analysis) where live blood not stained is analyzed by using a microscope, which is a method of directly observing erythrocytes, lymphocytes as leukocytes, granulocytes, macrophages or the like in the blood within a short time. Substantially circular shape means that the planar shape as observed by the microscope is substantially circular, and the examples of substantially circular shapes are shown in FIGS. 10, 12, 14, 16, 18 and 20. Separation means that individual erythrocytes are not agglomerated, and the examples in which erythrocytes are separated are shown in FIGS. 10, 12, 14, 16, 18 and 20.

The phrase “each erythrocytes in blood becomes substantially circular shape” means that all or most of the erythrocytes (preferably 90% or more) in blood become substantially circular shapes. The term “separate” includes the state where the erythrocytes of substantially circular shapes contact on a plane so that individual erythrocytes can be readily separated.

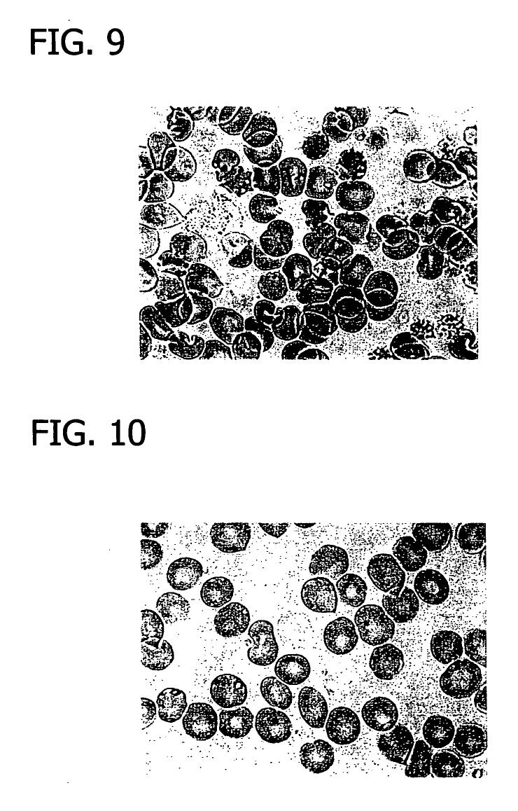

FIG. 9 shows the state where the erythrocytes not having substantially circular shapes are agglomerated to form an aggregated colony. Erythrocytes, which are separated as substantially circular shapes, are sized about 7 to 8 μm. Since the diameter of vascular cavity of capillary is approximately 5 μm, the blood in which erythrocytes are not separated as shown in FIG. 9 can hardly pass through the capillary vessel. On the contrary, normal blood where most of the erythrocytes are separated as substantially circular shapes according to the present invention readily passes through the capillary since it has deforming ability. As a result, arteriosclerosis owing to disturbance of blood flow, angina pectoris, cerebral thrombosis, brain infarct, pulmonary thromboembolism, myocardial infarction and intraatrial bleeding are prevented.

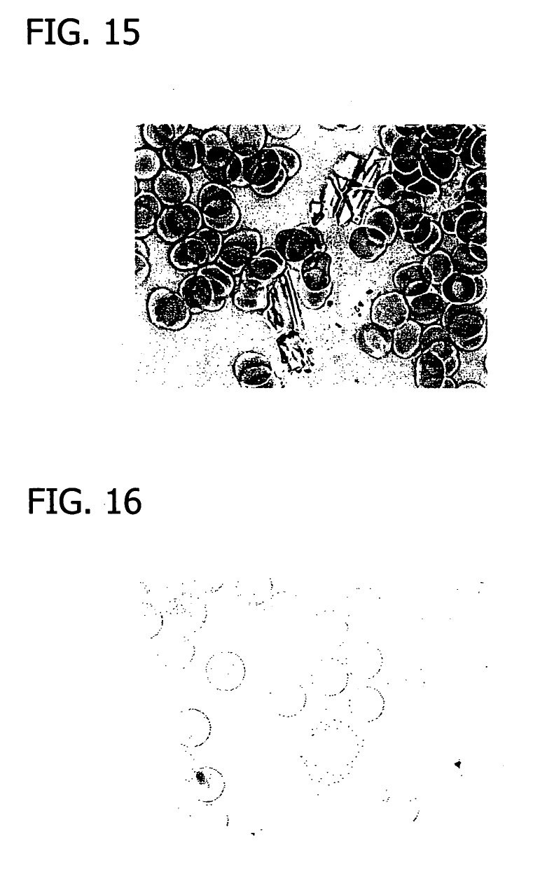

In addition, uric acid crystals (see FIG. 15), crystalline cholesterol and plaque in blood are dissolved by administration of the anti-tumor agent (fermented material) according to the present invention. Time period required for dissolution of uric acid crystals or the like is preferably within 90 days, more preferably within 60 days, and most preferably within 30 days from the administration. Such crystals of uric acid are considered causes of gout. If uric acid crystals contact the capillary vessel wall, one would feel severe pain. The pain can be eliminated in a relatively short amount of time such as within the time period mentioned above.

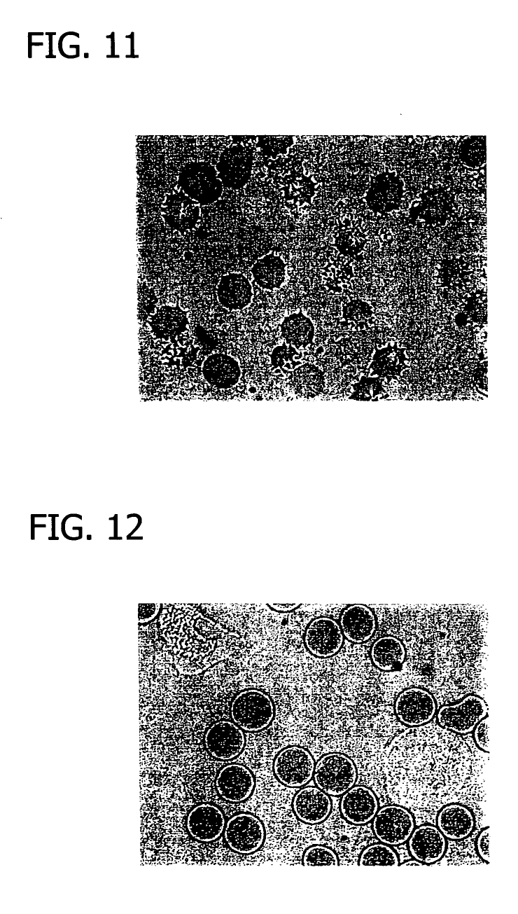

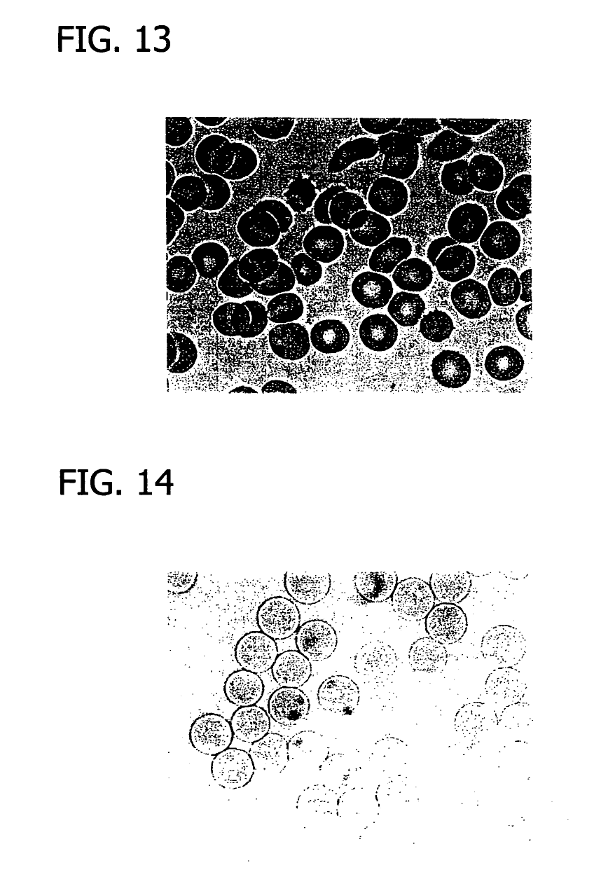

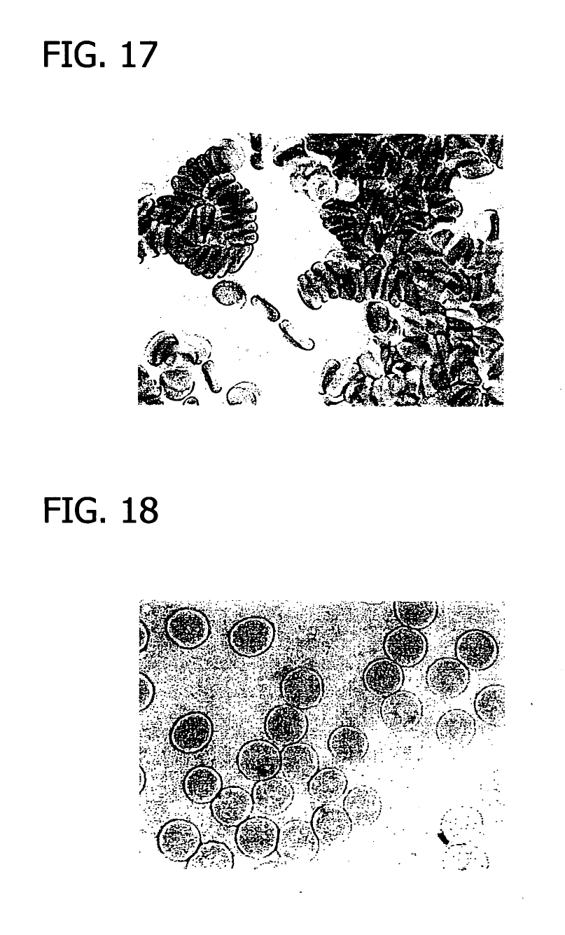

The anti-tumor agent (fermented material) according to the present invention, after being administered, restores acanthocytes (see FIGS. 9 and 11), target-cell type erythrocytes (see FIG. 13), erythrocytes arranged in chain (rouleau) (see FIG. 17), ovalocytes (see FIG. 13), free radical damaged erythrocytes (poikilocytosis) and the like into substantially circular shapes. It usually makes individual erythrocytes become separated. Normal erythrocytes have substantially circular shapes, while erythrocytes having other than substantially circular shapes such as acanthocytes can be called diseased or abnormal erythrocytes. The erythrocytes that deteriorate liver function are frequently acanthocytes. In case of anemia lacking iron content, there are many target cell type erythrocytes. In case of abnormal hormone secretion owing to thyroid abnormality or uterine myoma or the like, there are usually many ovalocytes.

FIGS. 10, 12, 14, 16, 18 and 20 show that each deformed abnormal erythrocyte became normal. The mechanism of how the deformed abnormal erythrocytes became normal has not been clearly proved. However, the administration of the anti-tumor agent (or fermented material prepared according to the process described above) of the present invention can provide one or more effect(s) selected from the group consisting of improvement of arthralgia owing to collagen disease, improvement of liver function, reduction of uterine myoma, reduction of cancer cells, decrease of cholesterol, decrease of triglycerides and treatment of atopic dermatitis.

Thus, the fermented material obtained by fermenting raw material containing one or more substance(s) selected from the group consisting of germs of wheat, germs of soybean and germs of rice with one or more microbes selected from the group consisting of leavens, yeasts and lactobacilli may be used for treating one or more diseases such as gout, various cancers, cerebral infarction, myocardial infarction, angina pectoris, collagen disease, anemia, abnormal hormone syndrome, hypercholesterolemia, hypertriglyceridemia, atopic dermatitis and the like, as well as being used as an anti-tumor agent.

The time required to make the abnormal erythrocytes such as acantocytes to normal erythrocytes is preferably within 120 minutes, more preferably within 60 minutes, and most preferably within 30 minutes from the administration. It is necessary to make the abnormal erythrocytes rapidly into normal erythrocytes. The anti-tumor agent (fermented material) according to the present invention makes abnormal blood normal in a short amount of time.

When observed by microscope according to Live Blood Analysis after administering the anti-tumor agent, the diameter of leukocytes in blood is preferably at least 2.2 times, more preferably at least 2.5 times, and most preferably at least 3.0 times that of normal erythrocytes. The size of leukocytes herein means the diameter of planar shape observed by microscope according to Live Blood Analysis.

In addition, the ratio of diameter of leukocytes in blood after administration of the anti-tumor agent to that before administration of the same agent (that is, diameter after administration/diameter before administration) is preferably 1.5 to 4, and more preferably 2.0 to 3.0. If the diameter of leukocytes is within this range, the activity of leukocytes increases to result in high immunity.

The anti-tumor agent according to the present invention increases the activity of leukocytes in blood to enhance immunity. It is known that leukocytes without activity have a small diameter and low mobility. From the observation by microscope according to Live Blood Analysis, it is shown that leukocytes in blood are enlarged and become well defined in shape after administration of the anti-tumor agent of the present invention, and particularly that macrophages move actively (see upper-left of FIG. 12). Further, among leukocytes, lymphocytes T cells and B cells which attack virus or cancer cells can be observed, and their sufficient activity can also be observed. FIG. 12 shows leukocytes, after the administration, which became enlarged so as to be 2.5 times the size of normal erythrocytes while being well defined in shape.

The dose of the anti-tumor agent according to the present invention is not particularly restricted. It may be administered depending on the body weight, age, sex or the like. For example, the dose is preferably from 1000 to 5000 mg/day, more preferably from 2000 to 4000 mg/day, and most preferably from 2500 to 3500 mg/day for an adult. The dose is usually divided to be administered 3 or 4 times per day. If it is below 1000 mg, then anti-tumor effects might not be sufficient. If more than 5000 mg is administered, then immunity against tumors corresponding to the amount is not obtained. As it originates from natural substances, the anti-tumor agent according to the present invention can be used very safely without adverse effects.

The anti-tumor agent of the present invention may contain one or more substances such as germs of wheat, whole rice, seaweed minerals, starch, vitamins and amino acids. In addition, one may drink or eat the anti-tumor agent of the present invention as it is. Alternatively, the agent may be added to beverages and foods. It may be also manufactured as health foods having a specific health effect, functional foods prepared to exhibit a function of controlling a living body, or health beverage formulations.

Process for Manufacturing the Anti-Tumor Agent

The process for manufacturing the anti-tumor agent described above is not particularly restricted, but it is essentially performed as follows:

The process according to the present invention comprises the steps of: (1) steaming one or more substance(s) selected from the group consisting of germs of wheat, germs of soybean and germs of rice at 50° C. to 150° C., or steaming the same after roasting the substance(s) at 50 to 150° C.; and (2) fermenting the resultant steamed product by using one or more microbes selected from the group consisting of leavens, yeasts and lactobacilli.

In the steaming step (1), as to the germs of wheat, germs of soybean and germs of rice, those described above for the anti-tumor agent may be used. The blending ratio of the raw materials as described above may also be applied.

The steaming process as described above may be applied. In addition, before the steaming, the germs of wheat or the like can be roasted at a temperature described above. The process for roasting that is described above can be also applied. In addition, in the fermenting step (2), leavens, yeasts and lactobacilli as described above can be also applied, and the fermenting process described above can be also applied. The fermented material prepared by the process described above can be employed in treatment of various diseases, as well as being used as an anti-tumor agent.

Further, the process may also comprise the step of treating the resultant fermented material with hot water. The temperature of the hot-water treatment is preferably from 60 to 100° C., more preferably from 65 to 98° C., and most preferably from 70 to 96° C. The treatment is carried out preferably for 5 to 35 minutes, more preferably for 10 to 30 minutes, and most preferably for 15 to 25 minutes. Occasionally, the anti-tumor agent, when treated with hot water, exhibits superior effects in comparison to the untreated agent.

Beverages and Foods Containing the Anti-Tumor Agent

(Beverages and Foods Containing the Fermented Material)

The beverages and foods containing the anti-tumor agent according to the present invention are characterized by using said anti-tumor agent (fermented material). “Beverages and foods containing the anti-tumor agent” according to the present invention includes beverage, solid food, or the like. Examples of types of beverages and foods include beverages and foods recognized with specific health effect, or functional beverages and foods manufactured in order to exhibit the function of components controlling the living body.

When the anti-tumor agent of the present invention is applied to beverages and foods, the anti-tumor agent can be manufactured as beverages or foods as it is, while the required amount of the same agent can be added to the raw material for beverages or foods to manufacture the beverages or foods containing the anti-tumor agent according to the conventional process. For example, the anti-tumor agent may be directly added to beverages or foods as an ingredient.

Alternatively, the anti-tumor agent may be dissolved in oils and fats, ethanol, propylene glycol, glycerin, surfactants or mixtures thereof to make liquid phase, or it may be added as a suspension to beverages and solid foods. If desired, it can be added to beverages and solid foods after being mixed with a binder such as Arabic gum, dextrin and the like to make powdery or granular type. The type of beverages and foods to which the anti-tumor agent of the present invention is added is not particularly restricted.

When the anti-tumor agent is added to beverages and foods, the amount of the agent is not particularly restricted, but the amount is properly adjusted depending upon the age, body weight and sex of the user. This is because ingestion of the agent as health food or functional food is intended to prevent diseases or to maintain health. Based on a total of 100% by weight of beverages and foods containing the anti-tumor agent, the amount of the agent is preferably from 1% to 30% by weight, more preferably from 2% to 25% by weight, and most preferably from 3% to 20% by weight.

### EXAMPLES

The present invention is now described with reference to the Examples illustrated below. In the Examples, the following conditions were employed.

Observation by microscopy according to Live Blood Analysis: Observation was carried out by using CH40 microscope (manufactured by Olympus) with magnification of 1000. Figures of erythrocytes referred in the following examples reflect the state observed by said microscope with magnification of 1000 (i.e., 1000 times the size of actual blood).

### Example 1

The blending ratio of raw materials was 250 g of germs of wheat, 250 g of germs of soybean and 200 g of germs of rice. The mixture was heated with steam at 110° C. for 3 hours, and then cooled. When the temperature became 30° C., the mixture was mixed with 250 g of wheat leaven and spread on a large vat. The mixture in said large vat was placed in a fermentation chamber to be fermented for 48 hours. Then, the vat was taken off and the mixture was dried at 40° C. for 48 hours to obtain an anti-tumor agent.

### Example 2

### Immunity Against Tumor In Vitro by Using Mice

(1) The anti-tumor agent obtained above was made as dry powder and dissolved in phosphate buffer. This was to manufacture the agent without hot water treatment and the agent was treated with hot water at 90° C. for 20 minutes (boiled). Each solution was added to cultured solution of human tumor cells A4573 and mouse melanoma B16 cells cultivated in 96-well plate. After 6 and 24 hours from the addition, the reagent for measuring the live cells (tetrazolium salt: manufactured by Nakarai Tesk) was added, and the color reaction was measured after 1 hour by using a microplate reader (450 nm). As to the control group without addition of the anti-tumor agent (hereinafter, simply referred to as “the control group”), identical measurement was performed. The measurement was repeated four times.

The results are shown in FIGS. 1 and 2. It is confirmed that the anti-tumor agent treated with hot water significantly lowered the number of live cells as compared to the control group in case of human tumor cells A4573. In case of mouse melanoma B16 cells, a significant decrease of number of cells as compared to the control group was confirmed.

(2) Solid feed containing 0.4% of the anti-tumor agent was prepared and provided to 5-week old C57BL6 mice as food. Daily dose of the anti-tumor agent ingested by a mouse with free intake of feed is estimated to be 10 times the dose of a man. Animal group fed with conventional solid feed was used as the control group. In order to evaluate the anti-tumor effects of the anti-tumor agent (1) against the subcutaneously implanted tumor, B16 cells (5×105 cells/mouse) were subcutaneously inoculated to 7 mice after 2 weeks from feeding. This was to measure the volume percentage of the tumor over time and to examine the survival rate after inoculation of tumor.

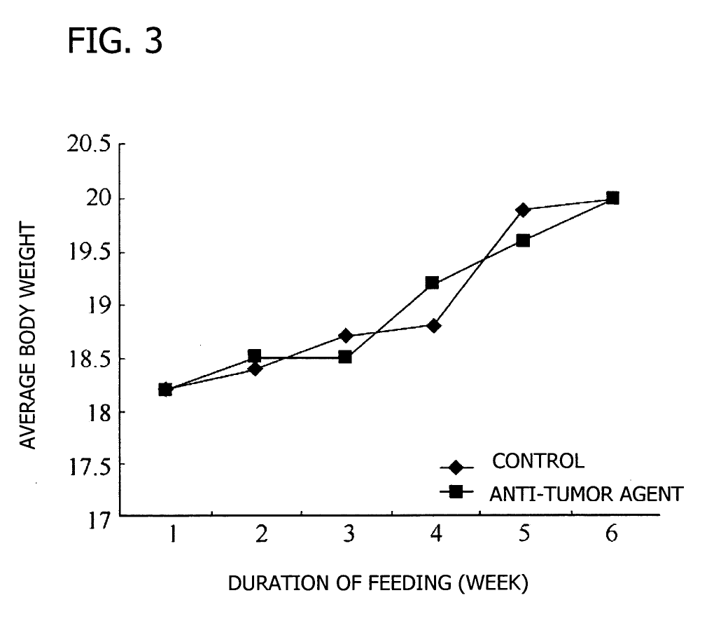

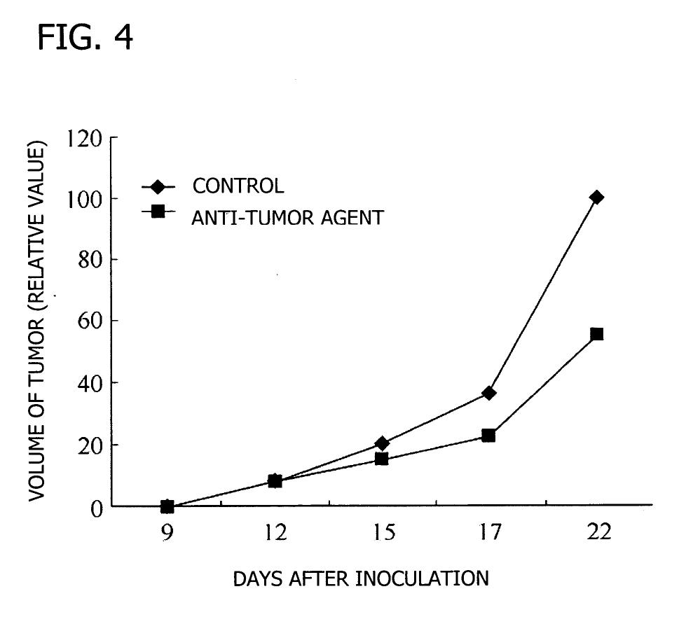

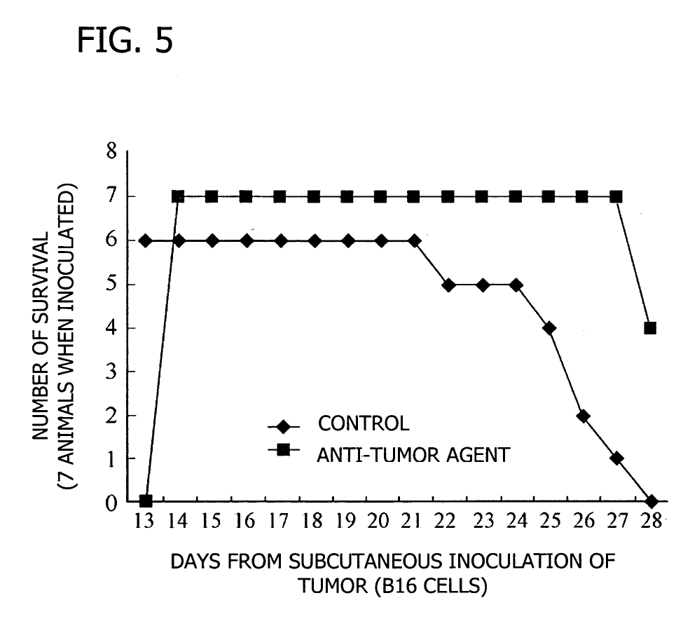

The results are shown in FIGS. 3, 4 and 5. In the mice with free intake of the feed, no significant change in the body weight was exhibited compared to the group fed with usual solid feed (FIG. 3). As such, it is suggested that a mouse equivalently ingests the feed containing the anti-tumor agent as usual feed. After two weeks of providing the feed, B16 cells (5×105 cells/mouse) were subcutaneously inoculated to 7 mice. As a result, excellent anti-tumor effects were confirmed since the volume percentage of the tumor of the group administered with the anti-tumor agent after 22 days from inoculation was 55% by volume of the control group (FIG. 4). In addition, as to the days of survival of the group inoculated with tumor, 4 mice among 7 mice of the group administered with the anti-tumor agent having subcutaneous inoculation of B16 cells (5×105 cells/mouse) survived, while no mice survived among the control group (FIG. 5).

(3) Solid feed containing 0.4% of the anti-tumor agent was prepared and provided to 5-week old C57BL6 mice as the feed.

Animal group fed with conventional solid feed was used as the control group. In order to evaluate the anti-tumor effects of the anti-tumor agent against transferable tumor, B16 cells (5×105 cells/mouse) are inoculated in caudal vein of 10 mice of each group after two weeks of providing said feed. Change in body weight over time was measured and lungs of half (5 animals) of each group were taken after 19 and 22 days from inoculation, to measure the number of cells of metastasis.

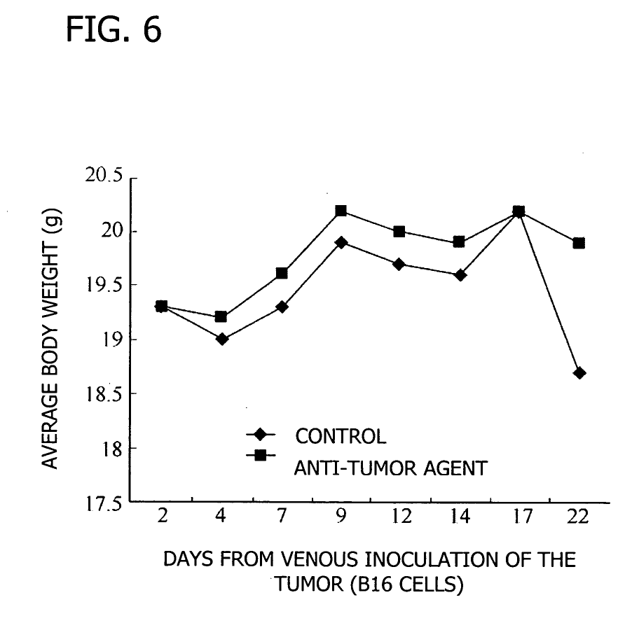

The results are shown in FIGS. 6, 7 and 8. As a result of measuring the change in body weight over time, the average body weight of the group administered with the anti-tumor agent of the present invention was 19.9 g, while average body weight of the control group was 18.6 g. The ratio of average body weight of the group administered with the anti-tumor agent of the present invention to that of the control group was 107% (FIG. 6).

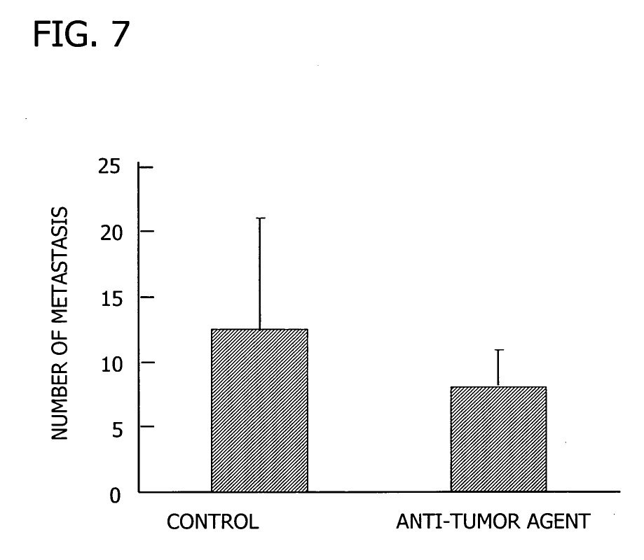

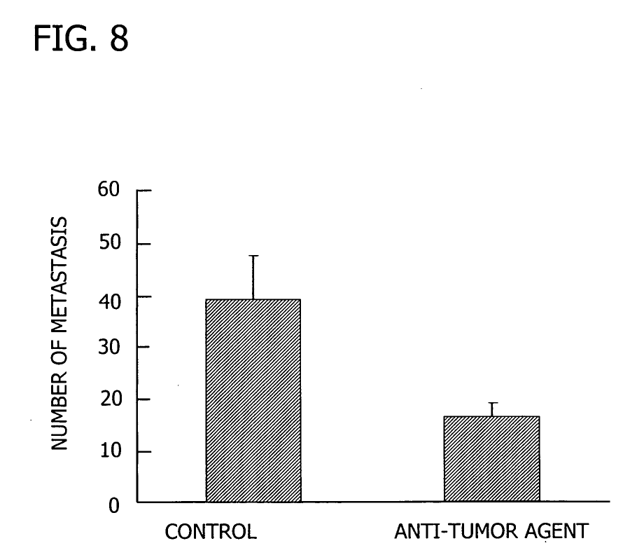

After 19 and 22 days from inoculation, lungs were taken from 5 animals of each group and the number of cells of metastasis was measured. The results are shown in FIGS. 7 and 8. After 19 days from inoculation, the average number of metastasis of the group administered with the anti-tumor agent according to the present invention was 8, while that of the control group was 12. The ratio of number of metastasis of the administered group to the control group was 66%. As such, the reduction in the number of metastasis of the former was confirmed. After 22 days from inoculation, the average number of metastasis of the group administered with the anti-tumor agent according to the present invention was 17, while that of the control group was 39. The ratio of number of metastasis of the administered group to the control group was 44%. Thus, the remarkable reduction in the number of metastasis of the former group was confirmed.

As described above, the metastasis of tumor cells is inhibited in mice administered with the anti-tumor agent.

### Example 3

### Observation by Microscope According to Live Blood Method

(1) Blood was taken from a female adult who was a patient of collagen disease. It was then observed by microscope according to Live Blood Analysis. Erythrocytes of the patient were agglomerated with irregular shape (FIG. 9). Liver function was not in a good condition and acanthocytes (spike type) were shown. The ability for glycolysis was not good and a lot of fungal forms could be seen. When the Live Blood Analysis was performed again after 30 minutes of administering 1170 mg of the anti-tumor agent, individual erythrocytes were separated as substantially circular shapes and no acantocyte (spike type) was found (FIG. 10).

To the patient, the same dose was continuously administered four times a day for 90 days. As a result, the characteristic arthralgia of collagen disease disappeared.

(2) Blood was taken from an adult female who was a patient having a disorder in liver function. It was then observed by microscope according to Live Blood Analysis. Numerous acanthocytes (spike type) were shown (FIG. 11). The patient had HCV antibody value of 88.9 S/CO (Reference of HCV antibody being 1.0 S/CO). After 30 minutes from administering 1170 mg of the anti-tumor agent, Live Blood Analysis was repeatedly performed. As a result, individual erythrocytes were separated as substantially circular shapes and no acantocyte (spike type) was found (FIG. 12). In addition, the diameter of all leukocytes shown in FIG. 12 became as large as 2.7 times as that of erythrocytes, which demonstrated increase of immunity. To the patient, the same dose was continuously administered three times a day for 90 days. As a result, the HCV antibody value became 10.4 S/CO, which is less than ⅛ of the value before the administration, thus exhibiting improvement in the liver function disorder.

(3) Blood was taken from a female adult who was a patient of uterine myoma. It was then observed by microscope according to Live Blood Analysis. Erythrocytes of this patient were ovoid and bad hormone balance could be recognized (FIG. 13). Target cell type erythrocytes having donut shape were seen, and it is recognized that she had anemia owing to iron deficiency. Hemoglobin value of this patient was 8.4 g/dl (normal value being from 11.5 to 15.5 g/dl). The anti-tumor agent (1170 mg) was administered to the patient. After 30 minutes from administration, blood was observed again by microscope according to Live Blood Analysis. As a result, individual erythrocytes were separated as substantially circular shapes and became normal state (FIG. 14). To the patient, the same dose was continuously administered three times a day for 90 days. As a result, uterine myoma of 3 cm in size was reduced to 1 cm. In addition, anemia was improved making hemoglobin values normal.

(4) Blood was taken from a female adult who was a patient of uterine cancer. It was then observed by microscope according to Live Blood Analysis. Erythrocytes of this patient were agglomerated, and fungi usually shown in case that uric acid crystals and sugar were not decomposed were observed (FIG. 15). The anti-tumor agent (1170 mg) was administered to the patient. After 30 minutes from administration, blood was observed again by microscope according to Live Blood Analysis. As a result, individual erythrocytes were separated as substantially circular shapes and uric acid crystals disappeared (FIG. 16). To the patient, the same dose was continuously administered three times a day for 90 days. As a result of MRI test and in vivo test, a diagnosis in which cancer disappeared was pronounced.

(5) Blood was taken from a male adult who had hyperlipidemia, hypercholesterolemia (261 mg/dl) and hypertriglyceridemia (1714 mg/dl). It was then observed by microscope according to Live Blood Analysis. Erythrocytes of this patient were erythrocytes arranged in chain with very bad agglomeration state. Further, leukocytes were so small and enclosed among erythrocytes that they cannot move by themselves. Blood flow was very poor to be in so-called “static blood” (in terms of Chinese medicine) (FIG. 17). The anti-tumor agent (1170 mg) was administered to him. After 30 minutes from administration, blood was observed again by microscope according to Live Blood Analysis. As a result, individual erythrocytes were separated as substantially circular shapes and recognized as normal (FIG. 18). The same dose was continuously administered three times a day for 120 days and blood test was carried out. Consequently, the cholesterol value became 227 mg/dl and triglyceride value was much lowered to become approximately normal (i.e., 169 mg/dl).

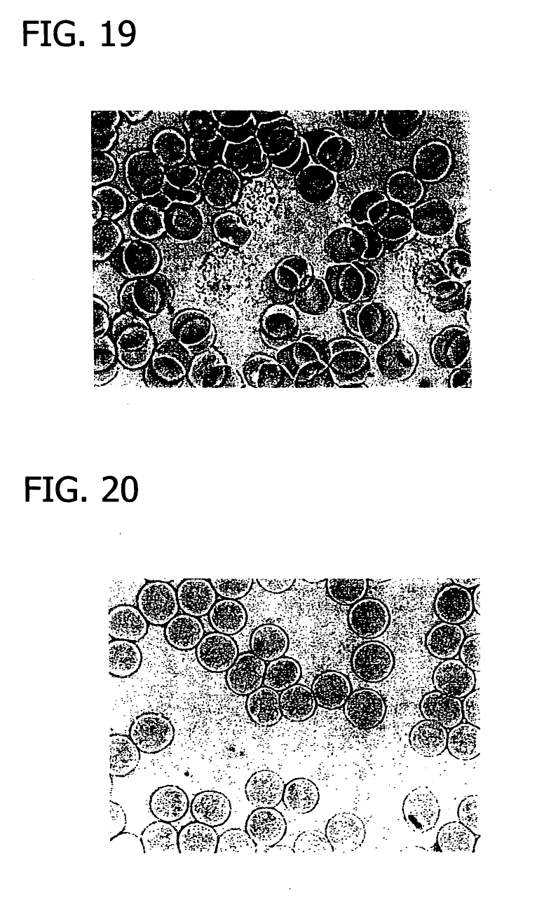

Blood was taken from a female adult who was a patient of atopic dermatitis. It was then observed by microscope according to Live Blood Analysis. Erythrocytes of this patient were agglomerated, and even three leukocytes (granulocytes) were observed in one image (FIG. 19). The existence of two or more granulocytes in one image is considered as allergic disease (such as asthma, rhinitis and pollenosis). The lesion already had inflammation. The anti-tumor agent (1170 mg) was administered to the patient. After 30 minutes from administration, blood was observed again by microscope according to Live Blood Analysis. As a result, individual erythrocytes were separated as substantially circular shapes, and the size of B cells of lymphocytes became 2.3 times as large as that of erythrocytes, being activated (FIG. 20). Since the lymphocytes were activated, it is thought that immunity was increased. To the patient, the same dose was continuously administered three times a day for 90 days. As a result, atopic dermatitis was completely healed.

As described above, it can be understood that the anti-tumor agent according to the present invention exhibits immune activity against tumors.

In addition, the fermented material obtained by fermenting raw material containing one or more substance(s) selected from the group consisting of germs of wheat, germs of soybean and germs of rice with one or more microbes selected from the group consisting of leavens, yeasts and lactobacilli may be used for treating one or more diseases such as gout, various cancers, cerebral infarction, myocardial infarction, angina pectoris, collagen disease, anemia, abnormal hormone syndrome, hypercholesterolemia, hypertriglyceridemia and atopic dermatitis.

Further, the anti-tumor agent according to the present invention shows immune activity against those other than tumors, as well as against tumors.

The present invention is not restricted to the specific Examples described above. Various modified examples can be made depending upon the purpose and use thereof within the scope of the present invention. Thus, instead of the anti-tumor agent, the fermented material obtained by fermenting raw material containing one or more substance(s) selected from the group consisting of germs of wheat, germs of soybean and germs of rice with one or more microbes selected from the group consisting of leavens, yeasts and lactobacilli may be directly applied to the Examples. Accordingly, said fermented material may be used to improve one or more diseases such as gout, various cancers, cerebral infarction, myocardial infarction, angina pectoris, collagen disease, anemia, abnormal hormone syndrome, hypercholesterolemia, hypertriglyceridemia, atopic dermatitis and the like.

The present invention is widely used for or as health beverage, health food, health auxiliary food, specific health food, medicines, and the like. For example, the anti-tumor agent according to the present invention may be added as it is or as beverages and foods with the same agent having been added. Further, the anti-tumor agent may be provided as medicines in the form of capsules, tablets, sachets, syrup, suppository, injectable preparations, or the like.

The anti-tumor agent according to the present invention has excellent anti-tumor effects. The anti-tumor agent according to the present invention, as coming from natural substances, shows high safety.

In addition, it makes erythrocyte aggregation, acanthocytes, target-cell type erythrocytes, erythrocytes connections, echinocytes, microcytes, macrocytes, hemolytic erythrocytes (hemolysis), erythrocytes arranged in chain (rouleau), ovalocytes and/or free radical damaged erythrocytes (poikilocytosis), having existed before internal use, into erythrocytes of substantially circular shapes with individual erythrocytes separated after internal use of the agent. In cases where it makes individual erythrocytes separated, the agent exhibits therapeutic effects on diseases due to erythrocytes of abnormal shapes, as well as showing excellent anti-tumor effects.

In cases where it makes the diameter of leukocytes in blood as large as 2.2 times that of normal erythrocytes after internal use and that granules in the granulocytes actively move, the anti-tumor effects are particularly prominent.

In addition, where it removes uric acid crystals in the blood, it provides relief from the pain caused by gout.

When showing one or more effects selected from the group consisting of improvement in arthralgia, improvement in liver function, reduction of uterine myoma, reduction of cancer cells, decrease of cholesterol, decrease of triglyceride and improvement in atopic dermatitis, it can be widely employed as health food or the like.

Beverages and foods according to the present invention have an excellent effect in preventing tumor, increasing immunity, and improving human health, while they can be used very safely without adverse effect since they come from natural origin.

According to the preparation process of the present invention, the anti-tumor agent can be simply and efficiently manufactured.

The anti-tumor agent obtained by a process for manufacturing the anti-tumor agent, which further comprises a step of treating with hot water at 60 to 100° C., exhibits excellent anti-tumor effects.

1. An anti-tumor agent obtained by fermenting raw material containing one or more substance(s) selected from the group consisting of germs of wheat, germs of soybean and germs of rice with one or more microbes selected from the group consisting of leavens, yeasts and lactobacilli.

2. The anti-tumor agent according to claim 1, wherein one or more types of erythrocytes, having existed before internal use, and selected from the group consisting of erythrocyte aggregation, acanthocytes, target-cell type erythrocytes, erythrocytes connections, echinocytes, microcytes, macrocytes, hemolytic erythrocytes (hemolysis), erythrocytes arranged in chain (rouleau), ovalocytes and free radical damaged erythrocytes (poikilocytosis), are restored into erythrocytes of substantially circular shape with individual erythrocytes separated after internal use of the agent.

3. The anti-tumor agent according to claim 1 or 2, wherein diameters of leukocytes in blood, after internal use, are 2.2 times or more than diameters of normal erythrocytes.

4. The anti-tumor agent according to claim 1 or 2, wherein uric acid crystals are removed from blood.

5. The anti-tumor agent according to claim 1 or 2, which exhibits one or more additional effect(s) selected from the group consisting of alleviating arthralgia, improving liver function, reducing uterine myoma, reducing cancer cells, decreasing cholesterol, decreasing triglyceride and treating atopic dermatitis.

6. Beverages and foods which contain the anti-tumor agent according to claim 1 or 2.

7. A process for manufacturing the anti-tumor agent according to claim 1 or 2, comprising the steps of (1) steaming one or more substance(s) selected from the group consisting of germs of wheat, germs of soybean and germs of rice at 50° C. to 150° C., or steaming the same after roasting the substance(s) at 50° C. to 150° C.; and (2) fermenting the resultant steamed product by using one or more microbes selected from the group consisting of leavens, yeasts and lactobacilli.

8. The process according to claim 7, further comprising the step of treating the resultant fermented material with hot water at 60° C. to 100° C.

9. The anti-tumor agent according to claim 3, wherein uric acid crystals are removed from blood.

10. The anti-tumor agent according to claim 3, which exhibits one or more additional effect(s) selected from the group consisting of alleviating arthralgia, improving liver function, reducing uterine myoma, reducing cancer cells, decreasing cholesterol, decreasing triglyceride and treating atopic dermatitis.

11. The anti-tumor agent according to claim 4, which exhibits one or more additional effect(s) selected from the group consisting of alleviating arthralgia, improving liver function, reducing uterine myoma, reducing cancer cells, decreasing cholesterol, decreasing triglyceride and treating atopic dermatitis.

12. Beverages and foods which contain the anti-tumor agent according to claim 3.

13. Beverages and foods which contain the anti-tumor agent according to claim 4.

14. Beverages and foods which contain the anti-tumor agent according to claim 5.

15. The process for manufacturing the anti-tumor agent according to claim 3, comprising the steps of (1) steaming one or more substance(s) selected from the group consisting of germs of wheat, germs of soybean and germs of rice at 50° C. to 150° C., or steaming one or more substance(s) selected from the group consisting of germs of wheat, germs of soybean, germs of rice and germs of whole rice after roasting the substance(s) at 50° C. to 150° C.; and (2) fermenting the resultant steamed product by using one or more microbes selected from the group consisting of leavens, yeasts and lactobacilli.

16. The process for manufacturing the anti-tumor agent according to claim 4, comprising the steps of (1) steaming one or more substance(s) selected from the group consisting of germs of wheat, germs of soybean and germs of rice at 50° C. to 150° C., or steaming one or more substance(s) selected from the group consisting of germs of wheat, germs of soybean, germs of rice and germs of whole rice after roasting the substance(s) at 50° C. to 150° C.; and (2) fermenting the resultant steamed product by using one or more microbes selected from the group consisting of leavens, yeasts and lactobacilli.

17. The process for manufacturing the anti-tumor agent according to claim 5, comprising the steps of (1) steaming one or more substance(s) selected from the group consisting of germs of wheat, germs of soybean and germs of rice at 50° C. to 150° C., or steaming one or more substance(s) selected from the group consisting of germs of wheat, germs of soybean, germs of rice and germs of whole rice after roasting the substance(s) at 50° C. to 150° C.; and (2) fermenting the resultant steamed product by using one or more microbes selected from the group consisting of leavens, yeasts and lactobacilli.
